(19) United States
(12) Patent Application Publication
(10) Pub. No.: US 2007/0065508 A1
(43) Pub. Date: Mar. 22, 2007

(54) Composition Comprising Vitamin B12 and Acid Reducing Agent and Uses Thereof

(75) Inventors: J. Mark Ruscin, Westminster, CO (US); Robert J. Valuck, Centennial, CO (US); Robert L. Page II, Denver, CO (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Regents of the University of Colorado, Boulder, CO (US)

(21) Appl. No.: 11/532,354

(22) Filed: Sep. 15, 2006

Related U.S. Application Data
(60) Provisional application No. 60/717,651, filed on Sep. 16, 2005.

(51) Int. Cl. A61K 31/714; A61K 31/495; A61K 31/4439; A61K 31/4172; A61K 9/20; A61K 33/00

(52) U.S. Cl. 424/464; 514/52; 514/338; 514/255.04; 514/400; 424/717

(57) ABSTRACT

The present invention provides compositions and methods for treating or reducing the risk of a vitamin B₁₂ deficiency in a subject having or in need of a gastrointestinal acid reduction treatment.

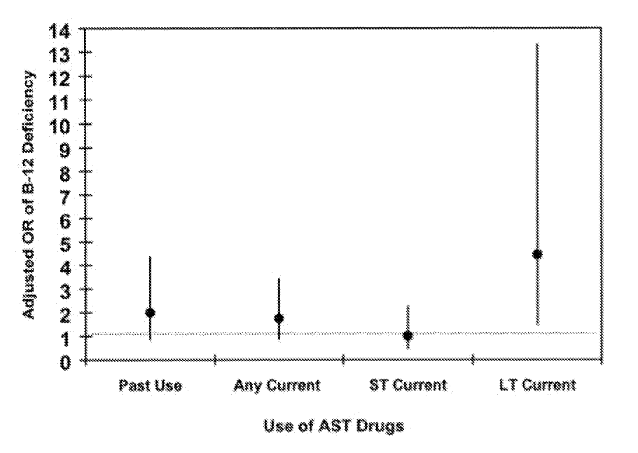

### CROSS-REFERENCE TO RELATED APPLICATIONS

This application claims the priority benefit of U.S. Provisional Application No. 60/717,651, filed Sep. 16, 2005, which is incorporated herein by reference in its entirety.

### FIELD OF THE INVENTION

The invention relates to a composition comprising a mixture of vitamin B₁₂ and a gastrointestinal acid reducing agent and methods for using the same.

### BACKGROUND OF THE INVENTION

Vitamin B₁₂ (cyanocobalamin) deficiency is a common disorder especially among older adults with the prevalence of metabolically confirmed deficiency estimated at 5-15% of the population older than 65. Vitamin B₁₂ deficiency has been associated with atherosclerosis, anemia, memory loss, psychological disorder, neurological degeneration and hyperhomocysteinemia including increased risk of heart disease, Alzheimer's disease, and osteoporosis related fractures.

Once a subject is deficient in B₁₂ some of the complications caused by the deficiency are not fully reversible with replacement of vitamin B₁₂ particularly the cognitive, psychological, and neurological complications. Therefore, preventing deficiency of vitamin B₁₂ in a subject before these affects take place would be an ideal approach.

A major source of vitamin B₁₂ for humans comes from animal foods in the diet, where B₁₂ is bound to protein. Vitamin B₁₂ must be released from dietary protein, a process that is believed to require gastric acid and pepsin, before it can be absorbed. Current research suggests that many older adults with vitamin B₁₂ deficiency do not adequately absorb food-bound vitamin B₁₂.

In certain situations, drugs such as acid blocking drugs have been shown to cause malabsorption of bound vitamin B₁₂ in subjects taking the drugs. This malabsorption can lead to vitamin B₁₂ deficiency. The availability of gastrointestinal medications over-the-counter to treat the symptoms of gastrointestinal acid conditions is on the rise and more and more people are consuming these medications without appropriate monitoring of side affects such as malabsorption of vitamin B₁₂.

In the last few years several gastrointestinal acid medications have gone from prescription to over-the-counter, for example many gastroesophageal reflux disease (GERD) medications include histamine2-receptor antagonists (H2As), proton-pump inhibitors (PPIs) and many more gastrointestinal drugs will be joining these readily available gastrointestinal medications. Thus, the occurrence of side affects created by the widespread use and potential overuse of these medications, such as vitamin B₁₂ malabsorption and deficiency, could escalate.

Thus, there is a need for therapies for vitamin B₁₂ supplementation generally, and reduction of the risk of onset of vitamin B₁₂ deficiencies in a subject using medications that reduce or inhibit the production of gastrointestinal acid.

### SUMMARY OF THE INVENTION

One aspect of the invention provides a composition comprising a mixture of vitamin B₁₂ and a gastrointestinal acid reducing agent.

In some embodiments, vitamin B₁₂ is not bound to protein.

In other embodiments, the gastrointestinal acid reducing agent comprises a gastrointestinal acid secretion reducing agent. Within these embodiments, in some instances the gastrointestinal acid secretion reducing agent is selected from the group consisting of a histamine2-receptor antagonist (H2RA), a proton-pump inhibitor (PPI), and a combination thereof In some cases, the PPI comprises omeprazole, lansoprasole, pantoprazole, rabeprazole, or a combination thereof In other cases, the H2RA comprises cimetidine, ranitidine, famotidine, nizatidine, or a combination thereof

In other embodiments, the gastrointestinal acid reducing agent comprises a gastrointestinal acid neutralizing agent. Within these embodiments, in some instances the gastrointestinal acid neutralizing agent comprises a bicarbonate.

Another aspect of the invention provides a method of reducing the risk of a vitamin B₁₂ deficiency in a subject having or in need of a gastrointestinal acid reduction treatment. The method comprises administering to a subject in need of such treatment, a therapeutically effective amount of a composition comprising a mixture of vitamin B₁₂ and a gastrointestinal acid reducing agent.

In some embodiments, vitamin B₁₂ is not bound to protein.

In other embodiments, the subject having or in need of a gastrointestinal acid reduction treatment has a condition selected from the group consisting of gastrointestinal acid reflux disease, gastroesophageal reflux disease, peptic ulcer disease, duodenal ulcer disease, Zollinger-Ellison disease, Hiatal hernia, esophageal erosions, esophagitis, Barrett's esophagus and esophageal strictures.

Still in other embodiments, the gastrointestinal acid reducing agent comprises a gastrointestinal acid secretion reducing agent. In some instances within these embodiments, the gastrointestinal acid secretion reducing agent comprises a histamine2-receptor antagonist (H2RA), a proton-pump inhibitor (PPI), or a combination thereof.

Yet in other embodiments, the gastrointestinal acid reducing agent comprises a gastrointestinal acid neutralizing agent. In some instances, the gastrointestinal acid neutralizing agent comprises a bicarbonate.

In other embodiments, the subject is a human.

### BRIEF DESCRIPTION OF THE DRAWINGS

The FIGURE is a graph showing adjusted odds ratio of B₁₂ deficiency by exposure to acid suppressant therapy (AST) drugs.

### DETAILED DESCRIPTION OF THE INVENTION

For illustrative purposes, embodiments of the present invention are described with reference to vitamin B₁₂ deficiencies in humans. However, it will be appreciated by those skilled in the art that the embodiments described herein can be applied to other nonhuman subjects, such as other mammals, at risk of a vitamin B₁₂ deficiency.

One aspect of the invention provides a composition comprising a mixture of vitamin B₁₂ and a gastrointestinal acid reducing agent. In some embodiments, the vitamin B₁₂ is not bound to protein, i.e., vitamin B₁₂ is free vitamin B₁₂. The gastrointestinal acid reducing agent can include, but is not limited to, a gastrointestinal acid secretion reducing agent, a gastrointestinal acid neutralizing agent, and a combination thereof Exemplary gastrointestinal acid secretion reducing agents include, but are not limited to, a histamine2-receptor antagonists (H2RA), a proton-pump inhibitors (PPI), and a combination thereof Exemplary gastrointestinal acid neutralizing agents include, but is not limited to, a bicarbonate such as sodium bicarbonate, magnesium bicarbonate, calcium bicarbonate, and a mixture thereof.

Another aspect of the invention provides methods of treating and/or reducing the risk of the onset of a vitamin B₁₂ deficiency mediated disorder in a subject in need of or having a treatment to reduce gastrointestinal acid. Methods of the invention are suitable for, but not limited to, treating humans at risk of developing a vitamin B₁₂ deficiency due to use of drugs including, but not limited to, drugs that suppress gastrointestinal acid production or neutralize gastrointestinal acid such as gastrointestinal acid reducing agents. Drugs that suppress gastrointestinal acid production are well known to one skilled in the art and include prescription and over-the-counter drugs or combinations thereof Typically, methods of the invention include treating and/or reducing the risk of developing a vitamin B₁₂ deficiency in a subject in need of or having a treatment for a gastrointestinal acid mediated condition by administering to the subject a therapeutically effective amount of a mixture comprising vitamin B₁₂ and a gastrointestinal acid reducing agent. In one embodiment, methods of the invention include treating and/or reducing the risk of developing a vitamin B₁₂ deficiency in a subject in need of or having a treatment for a gastrointestinal acid mediated condition by administering to the subject a therapeutically effective amount of a composition comprising a mixture of vitamin B₁₂ and a gastrointestinal acid reducing agent. Such a mixture can be a single pill, capsule, caplet or single liquid dose or dosage form.

As used herein “mixture” refers to a single solid or liquid dose that comprises two or more different ingredients, e.g., both vitamin B₁₂ and a gastrointestinal acid reducing agent in a single dosage form. The term “mixture” often refers to one or more agents that are blended or are intimately distributed within a composition (e.g. vitamin B₁₂ and a gastrointestinal acid reducing agent) such as a single pill or a single liquid dose for use in a therapeutic pharmaceutical application. Therefore, a composition comprising a mixture of vitamin B₁₂ and a gastrointestinal acid reducing agent refers to a mixture of vitamin B₁₂ and a gastrointestinal acid reducing agent in a single dosage form, including those that are blended together or are intimately distributed within one another. For example, vitamin B₁₂ and a gastrointestinal acid reducing agent are contained within a single dosage form (e.g. tablet, capsule, and caplet). Accordingly, the term “mixture” does not include a separate composition that are administered together.

“A therapeutically effective amount” or “effective amount” means the amount of a compound or composition that, when administered to a subject for treating a disorder, is sufficient to effect such treatment for the disorder. The “therapeutically effective amount” can and will most likely vary depending on the compound or the composition, the disorder and its severity and the age, weight, etc., of the subject to be treated.

“Treating” or “treatment” of a disorder includes: (1) preventing the disorder, i.e., causing the clinical symptoms of the disorder not to develop in a subject; (2) inhibiting the disorder, i.e., arresting or reducing the development of the disorder or its clinical symptoms; or (3) relieving the disorder, i.e., causing regression of the disorder or its clinical symptoms.

All references, patents and publications sited in this disclosure are incorporated herein by reference in their entirety.

Any known vitamin B₁₂, vitamin B complex, B₁₂ precursors including, but not limited to, cobalamin, cyanocobalamin, hydroxycobalamin, or any other natural or synthetic form, can be used in the compositions of the invention. Often vitamin B₁₂ that is not bound to protein is used in compositions of the invention. Vitamin B₁₂ not bound to protein refers to a vitamin B₁₂ that does not require liberation from a food source, more particularly liberation from a protein molecule prior to absorption by the small intestine or gastrointestinal tract. In particular, “vitamin B₁₂ not bound to protein” refers to vitamin B₁₂ that does not require liberation from a protein molecule by gastrointestinal acid.

In some embodiments, subjects affected by the composition of the invention can be a subject suffering from a disorder that contributes to gastrointestinal acid production and this acid production is being controlled by a medication.

Still in other embodiments, the subject treated by the composition of the invention can be, but are not limited to, a subject suffering from gastrointestinal acid mediated disorders such as ulcers (peptic ulcer disease including the treatment or prevention of ulcer disease related to the use of non-steroidal anti-inflammatory drugs or NSAIDS), Zollinger-Ellison Disease, gastroesophageal reflux disease (GERD) or nonspecific gastrointestinal acid mediated disorders, or combination thereof that contribute to the production of gastrointestinal acid causing the subject to take a prescription and/or over-the counter medications to decrease the amount of or neutralize the gastrointestinal acid produced.

Without being bound by any theory, it is believed that one of the adverse effects of taking a gastrointestinal acid reducing agent is reduced gastrointestinal acid available for vitamin B₁₂ release from food sources, and therefore the amount of vitamin B₁₂ absorption.

Some embodiments of the invention include administering to a subject suffering from a gastrointestinal acid mediated disorder, a therapeutically effective amount of a composition comprising a mixture of vitamin B₁₂ and a histamine2(H2)-receptor antagonist. Generally, the H2RA are known to reduce gastric acid secretion into the stomach and intestine. Exemplary histamine2 (H2)-receptor antagonists include, but are not limited to, cimetidine, ranitidine, famotidine and nizatidine.

Other embodiments of the invention include administering to a subject suffering from a gastrointestinal acid mediated disorder, a therapeutically effective amount of a composition comprising a mixture of vitamin B₁₂ and a proton-pump inhibitor. Exemplary proton-pump inhibitors include, but are not limited to, omeprazole, lansoprasole, pantoprazole, and rabeprazole.

Vitamin B₁₂ Deficiency

The majority of vitamin B₁₂ for humans comes from animal sources in the diet (meat, fish, eggs, milk). Typically, this vitamin is bound to protein in animal food sources. In the presence of gastric acid, vitamin B₁₂ is freed from protein before it is absorbed in the small intestine.

Drug Induced

Vitamin B₁₂ deficiency can arise as a consequence of multiple factors. For instance, one mode leading to vitamin B₁₂ deficiency includes drug-induction of vitamin B₁₂ deficiency. Vitamin B₁₂ deficiency is a relatively common disorder among older adults, with the prevalence of metabolically confirmed B₁₂ deficiency estimated at 5 to 15% in people over 65 years of age. Although there are multiple etiologies for vitamin B₁₂ deficiency, it is believed that pernicious anemia is the cause in only a small percentage of new diagnoses in screening studies of older patients. The majority of older adults with vitamin B₁₂ deficiency are found to have normal intrinsic factor functioning with a normal Schilling test, but malabsorb dietary protein-bound vitamin B₁₂.

Without being bound by any theory, this is believed to be related to the development of atrophic gastritis and hypochlorhydria/achlorhydria with advancing age, and possibly other factors such as Helicobactyer pylori infection. Postgasrectomy states, atrophic gastritis, pancreatic insufficiency, and ileal disease are among some of the problems that is believed to lead to vitamin B₁₂ malabsorption. However, the human liver typically stores adequate vitamin B₁₂ so that deficiency states may take many months to years to develop, even in the face of vitamin B₁₂ malabsorption. It should be noted that vitamin B₁₂ deficiency is not a benign occurrence, because neurologic and psychiatric symptoms may not be fully reversible even with adequate treatment.

Histamine H-2 receptor antagonists (H2RA) and proton pump inhibitors (PPI) are widely used for their therapeutic effects related to reducing gastric acid production. Since the introduction of H2RA, and subsequently the PPIs, these agents have become one of the most frequently prescribed medications in the United States. For example, in 2000, omeprazole and lansoprazole were, respectively, the first and third most prescribed medications in the United States in retail sales. Although these medications were initially approved for short-term use in patients with peptic ulcer disease (PUD) and gastroesophageal reflux disease (GERD), their use as chronic maintenance therapy in PUD, GERD, and for ulcer prophylaxis with nonsteroidal anti-inflammatory agents is becoming increasingly common.

Additionally, many older patients are placed on these medications long term for nonspecific gastrointestinal complaints and are infrequently evaluated for continued need of these medications. It is believed that the use of medications that suppress gastric acid production produce malabsorption of dietary protein-bound vitamin B₁₂.

The present inventors have found that older patients with vitamin B₁₂ deficiency were significantly more likely to have used a gastrointestinal acid reducing agent, such as H2RA or PPI, for a prolonged period, e.g., 12 months or more. It is becoming increasingly common for patients to take a gastrointestinal acid reducing agent on an extended basis to prevent recurrent gastroesophageal reflux (GER) symptoms, to avoid potential GER complications such as peptic stricture and Barrett's esophagus, and for prophylaxis against NSAID induced gastropathy.

Although the H2RAs and PPIs are generally considered to be safe, it is believed that these medications promote the malabsorption of food-bound vitamin B₁₂. Older patients are already at increased risk for vitamin B₁₂ deficiency compared to younger populations, and may be more likely to have borderline vitamin B₁₂ status at baseline. Therefore, older patients are believed to be at greater risk of developing vitamin B₁₂ deficiency with additional factors, such as H2RA/PPI use, that can influence vitamin B₁₂ absorption.

Accordingly, another aspect of the invention provides a composition comprising a mixture of vitamin B₁₂ and a gastrointestinal acid reducing agent. Such compositions are useful, among others, in reducing the risk of a vitamin B₁₂ deficiency in a subject having or in need of a gastrointestinal acid reduction treatment. Drug-induced gastric acid suppression due to use of histamine2(H2)-receptor antagonists or proton-pump inhibitors (PPIs) cause malabsorption of protein-bound vitamin B₁₂. Without being bound to any theory, this malabsorption is believed to be due to insufficient amount of gastrointestinal acid necessary to cleave a sufficient amount of vitamin B₁₂ from protein in food sources. It is known that vitamin B₁₂ need to be cleaved from protein in order to be efficiently absorbed in the stomach. It is also known that gastric acid aids in cleavage of vitamin B₁₂ from protein. Since H2 receptor antagonists and PPIs reduce gastric acid production, it is believed that their use, in particular prolonged use (i.e., 12 month or longer), reduces absorption of vitamin B₁₂ from the food sources. H2RAs and PPIs are among the most frequently prescribed medications in the U.S. and are often taken for long periods of time.

In some embodiments of the invention, a composition includes a mixture of vitamin B₁₂ and an agent capable of neutralizing gastric acid such as an antacid. In some instances within these embodiments, the composition comprises a mixture of vitamin B₁₂ and an antacids, such as bicarbonates, carbonates, and hydroxides. Exemplary antacids include, but are not limited to, magnesium bicarbonate, aluminum bicarbonate, magnesium hydroxide, aluminum hydroxide (e.g., Maalox®, Mylanta®), sodium bicarbonate, potassium bicarbonate and calcium carbonate/bicarbonate (e.g., Tums® and Rolaids®).

In compositions of the invention, any vitamin B₁₂ that does not require gastric acid release for intestinal absorption can be used. These forms of vitamin B₁₂ are readily absorbed by the small intestine or gastrointestinal tract. It should be appreciated that even if the subject has a deficiency of intrinsic factor which is a co-factor needed for the active absorption of B₁₂ in the terminal ileum, the patient can absorb some free vitamin B₁₂ by mass action (non-active transport).

There are a variety of assay methods to determine whether a particular compound is capable of absorption by a subject and a variety of methods to measure the level of uptake of vitamin B₁₂ in a subject. Therefore a subject can be readily monitored for levels of vitamin B₁₂ anytime before, during or after a treatment using any of the embodiments of the invention.

In other embodiments of the invention, the composition comprises a mixture of vitamin B₁₂ and a gastrointestinal acid reducing agent, for example, a histamine-2 (H2) receptor antagonist (H2RA) and/or a proton-pump inhibitor. H2RA are widely used to treat gastrointestinal acid disorders. In certain cases, cimetidine, as well as, other agents capable of suppressing acid production is taken on a chronic basis to prevent gastroesophageal reflux disease (GERD) symptoms and to avoid potential complications, such as peptic stricture and Barrett's esophagus and complications related to non-steroidal anti-inflammatory drugs. In certain cases, these agents are prescribed for extended periods of time leading to a higher risk of vitamin B₁₂ deficiency. Alternatively, in some cases the onset of malabsorption of vitamin B₁₂ occurs early on in the regiment of taking these medications but symptoms of malabsorption can take months to manifest. Some of the commercially available H2RAs include Zantac® (GlaxoSmithKline), Pepsid (OTC, Over the counter), Axid® (Lilly) and Tagamet (GlaxoSmithKline). These medications can be administered orally as a solid or a liquid or introduced intravenously. Some of the compositions of the invention include mixtures that comprise one or more of currently commercially available H2RAs and vitamin B₁₂, often vitamin B₁₂ that is not bound to a protein.

Proton-pump inhibitors (PPIs) are also known to suppress the production of gastric acid. PPIs are available by prescription and over-the-counter, for example, Prilosec® and over the counter (OTC) Prilosec , which is one of the best selling prescription drugs in the world (Astra-Merck).

Other disorders that can be treated by the compositions of the invention include, but are not limited to, complications due to the gastrointestinal acid affects related to the use of non-steroidal anti-inflammatory drugs, Hiatal hernia, Barrett's esophagus, esophageal stricture, esophagitis, and esophageal erosions.

Compositions of the invention can be used to treat or prevent the onset of a vitamin B₁₂ deficiency in infants, children and adult subjects as well as other mammals.

Methods of Detection of Vitamin B₁₂ in a Subject

Generally, a test to measure the level of vitamin B₁₂ in a subject requires obtaining a fluid, e.g., blood, sample and detecting the level of vitamin B₁₂ in the fluid by any methods known in the art. The level of vitamin B₁₂ in a subject can also be measured by obtaining a tissue or cell sample and detecting the level of vitamin B₁₂ in the tissue or cells by any of the methods known in the art. The level of vitamin B₁₂ can be measured directly or indirectly, such as by measuring methylmalonic acid (MMA) and/or and homocysteine (HCYS) test levels.

Another aspect of the invention include a method for detecting the presence or absence of a vitamin B₁₂ deficiency mediated disorder. For example, pernicious anemia can be measured using the Schillings test. Alternatively, the level of anti-intrinsic factor antibodies can be measured, which is an indicator of pernicious anemia. Intrinsic factor aide in the active absorption of vitamin B₁₂ by the small intestine; therefore, decreased amount of intrinsic factor is an indication of reduced vitamin B₁₂ absorption.

In some instances, it is useful to compare levels of vitamin B₁₂ in a subject treated with a composition of the invention to levels of vitamin B₁₂ in a subject treated with a different composition of the invention or not treated with a composition of the invention. A subject screened for vitamin B₁₂ levels is generally considered deficient in vitamin B₁₂ if a sample such as a serum sample is about 200 pg/ml or less. In addition, if the level of vitamin B₁₂ is low (e.g., about 350 pg/mL or less, often about 200 to about 350 pg/ml) and/or if other tests known in the art associated with a vitamin B₁₂ deficiency, such as methylmalonic acid (MMA) tests and homocysteine (HCYS) test levels are elevated, the subject can be considered deficient in vitamin B₁₂.

Formulations

Compositions described herein can be administered to a subject to achieve a desired physiological effect. Generally the subject is an animal, often a mammal, and more often a human. The composition can be administered in a variety of forms adapted to the chosen route of administration, e.g., orally.

The compositions of the invention can be orally administered, for example, with an inert diluent or with an assimilable edible carrier, or it can be enclosed in hard or soft shell gelatin capsules, or it can be compressed into tablets, or it can be incorporated directly with the food of the diet, for example infant formula. For oral therapeutic administration, the composition can be incorporated with excipient and used in the form of a single ingestible tablet, buccal tablet, troche, capsule, elixir, suspension, syrup, wafer, and the like. Such compositions and preparation can contain at least 0. 1% each of vitamin B₁₂ and a gastrointestinal acid reducing agent. The percentage of vitamin B₁₂ and a gastrointestinal acid reducing agent and the mixture's preparation can be varied and can conveniently be between about 1 to about 10% of the weight of the unit. The therapeutically useful amount of vitamin B₁₂ and a gastrointestinal acid reducing agent is such that a suitable dosage is obtained. Generally compositions or preparations according to the invention are prepared such that an oral dosage unit form contains from 1 mcg to about 1000 mcg of the daily recommended dosage of vitamin B₁₂ and about 1 mg to about 1000 mg of the gastrointestinal acid reducing agent.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain any of the contemplated compositions of the invention, sucrose as a sweetening agent, methyl and propylparabens preservatives, a dye and flavoring such as cherry or orange flavor. Of course, any material used in preparing any dosage unit form should be pharmaceutically pure and substantially non-toxic in the amounts employed.

Compositions of the invention can be administered to a subject alone or in combination with pharmaceutically acceptable carriers, as noted above, the proportion of which is determined by, among others, chosen route of administration. A physician can readily determine the dosage of each components (e.g., vitamin B₁₂ and a gastrointestinal acid reducing agent) of the composition which is most suitable for prophylaxis, treatment or reduction of risk of onset and it can vary with the form and the particular composition chosen, and also, it can vary with the particular patient under treatment. The physician will generally wish to initiate treatment with a dose containing a level of vitamin B₁₂ where the optimum effect under the circumstances is reached. The dosage of vitamin B₁₂ component of a composition can generally be from about 0. 1 microgram (mcg) to about 10,000 mcg/dose, typically from about 5 to about 2000 mcg/dose, and often from about 10 to about 1000 mcg/per dose. In addition, the physician will generally wish to initiate treatment with a level of gastrointestinal acid reducing agent where the desired effect under the circumstances is reached. The therapeutic dosage of a gastrointestinal acid reducing agent of compositions of the invention can generally be from about 0.1 mg to 500 mg per dose, often from about 10 to about 250 mg per dose, and more often from about 20 to about 200 mg per dose. In one exemplary application, a single dose, such as a single pill, of PPI (e.g., omeprazole) and vitamin B₁₂ can comprise 10-40 mg and 500-1000 mcg, respectively. In another exemplary application, a single dose, such as a single pill, of H2A (e.g., ranitidine) and vitamin B₁₂ can comprise 75-300 mg and 500-1000 mcg, respectively.

Additional objects, advantages, and novel features of the present invention will become apparent to those skilled in the art upon examination of the following examples thereof, which are not intended to be limiting.

### EXAMPLES

Vitamin B₁₂ Deficiency Mediated Disorders

### Example 1

Oral vitamin B₁₂ has been studied as a treatment and preventive measure and has been shown to be an inexpensive, and effective intervention. Oral administration typically ranged from doses of 5-1000 mcg daily, as well as high doses (>1000 mcg, for example, 2500 to 5000 mcg) given daily. Thus, the additional vitamin B₁₂ is one solution to prevent the sometimes irreversible effects of vitamin B₁₂ deficiencies.

Oral doses around 1000 mcg can be administered to a subject taking or in need of taking gastrointestinal acid reducing agents such as PPI (e.g., omeprazole) and the level of vitamin B₁₂ measured before and/or after consumption of the supplement. In one example study, by 24 patients (12 per group) to either omeprazole 40 mg daily+oral placebo, or omeprazole 40 mg daily+oral vitamin B₁₂ 1000 mcg daily for four weeks. Omeprazole and vitamin B₁₂ supplements are dosed concurrently. Vitamin B₁₂ concentrations can be compared between baseline and the end of study period, expressed as percent change relative to baseline. In addition, influences on the pharmacokinetics of omeprazole is determined for a composition comprising omeprazole and vitamin B₁₂ 1000 mg. This can be achieved by comparing pharmacokinetlcs (AUC, Cₘₐₓ, Tₘₐₓ) of omeprazole 40 mg daily alone to omeprazole 40 mg daily+oral placebo, and/or omeprazole 40 mg daily+oral B₁₂ 1000 mg.

### Example 2

Subjects are given omeprazole 40 mg daily alone for 13 days (study day #1-#13). On day 14, subjects return to clinic and have a venous catheter placed to allow for serial blood draws for baseline omeprazole pharmacokinetic studies. Subjects are given the day 14 dose of omeprazole in the clinic and blood draws (5 ml) for pharmacokinetic analysis can occur predose (time 0) and at 8 additional time points (20, 40, 60, 90, 120, 180, 360, and 480 minutes) postdose. Subjects are then randomized (in single/double blind fashion) to receive either oral placebo daily or oral vitamin B₁₂ 1000 mg daily to be taken concurrently with omeprazole 40 mg daily for the remainder of the study period (day # 15-day #42), Compliance with study medications is evaluated by self-report and pill counts during study visits. At the end of week 4 (study day #28), subjects return to clinic to have blood drawn for repeat pharmacokinetic studies. Subjects are given the day 28 dose of omeprazole+either oral placebo or oral B₁₂ 1000 mg, depending on group assignment, and blood drawn at multiple time points for evaluation of B₁₂ levels.

Samples from the subjects are collected, via an indwelling venous catheter, in evacuated test tubes containing sodium heparin, refrigerated for five minutes at 4° C., and then centrifuged for 10 minutes at 2400 rpm. The plasma are stored at −80° C. until analysis. The omeprazole concentrations are determined using a high-performance liquid chromatographic (HPLC) method or any known method in the art. The HPLC system consists of a solvent-delivery pump (Beckman Coulter model 118), autosampler (Spectra-Physics model 880), variable-wavelength ultraviolet detector (Beckman Coulter model 166), column (Waters C18 Bondapak), computer software for data collection and analysis (Beckman Gold system), and solvent recycler (Adtech model 2000). The assay utilizes a mobile phase consisting of acetonitrile and 0.05 M (pH 8.1) phosphate buffer (25:75). The flow rate and absorption wavelength are set at 1 ml/min and 302 nm, respectively.

Frozen samples are allowed to thaw at room temperature. The internal standard (phenacetin, 30 fxl of 100-jAg/ml stock solution in methanol), 0.25 ml of 0.5 M phosphate buffer (pH 8.0), and 3 ml of dichloromethane are added to 1.0 ml of the plasma sample in a capped tube. After 40 minutes of agitation, the sample is centrifuged for 10 minutes at 2600 rpm. The upper aqueous layer are discarded, and the organic phase transferred to another test tube and dried under nitrogen gas at 40° C. The residue can reconstitute into 150 μl of mobile phase. Fifty microliters of the reconstituted solution are injected into the HPLC column. Peak height ratios of omeprazole to the internal standard are used to calculate plasma omeprazole concentrations. Intraday coefficients of variation at omeprazole concentrations of 50 and 400 ng/ml are 4.5% and 2.0%, respectively. The interday coefficients of variation at 50 and 400 ng/ml are 6.8% and 3.7%, respectively.

The plasma omeprazole concentrations and the sampling times for each subject are fitted, using a noncompartmental pharmacokinetic model. Additional pharmacokinetic modeling are performed once the initial non-compartmental analyses are completed. Pharmacokinetic values, including, Cₘₐₓ, tₘₐₓ and AUCo-∞ (where t is the time of the last sample collection, or 480 min), and AUCo-∞ (where ∞ is infinity) are determined.

### Example 3

A composition comprising PPI and vitamin B₁₂ (referred to as PPI/B₁₂) is administered to a subject without a gastrointestinal acid disorder or pancreatic disease. The subject, a 36 year old Caucasian male had no known history of gastrointestinal or pancreatic disease and no known history of vitamin deficiency or malabsorptive syndromes. He was not a vegetarian and took no prescription medications (OTC or prescription), supplements (including multivitamins), or herbal remedies. The subject began eating a strict vegetarian diet on Feb. 3, 2005, with no allowed supplements, including multivitamins, nutrition bars, nutrition drinks, fortified grain products such as cereals, or other supplements that might contain free (i.e., non-protein bound) vitamin B₁₂. The subject had a baseline serum vitamin B₁₂ test performed on one morning, which was found to be 476 pg/ml (nml 200-1100 pg/ml). On the evening of the same day, the subject began taking lansoprazole 30 mg twice daily (alone) for 3 days (72 hours). This allowed time for the lansoprazole to fully work and to produce a hypochlorhydric/achlorhydric (reduced/absence of gastric acid) state. Three nights later, the subject began taking combined PPI B₁₂ twice daily (each capsule contained lansoprazole 30 mg and cyanocobalamin 500 mcg). The subject continued the strict vegetarian diet, as described above, and the combined PPI B₁₂ product until 15 full days later. The subject returned for a repeat serum vitamin B₁₂ test on the morning of the 15th day, which was found to be 591 pg/ml (nml 200-1100 pg/ml, same lab was used as the first assay). The use of vitamin B₁₂ supplement while using a gastrointestinal acid reducing agent, such as the PPIs, resulted in increased vitamin B₁₂ absorption and an increase in serum vitamin B₁₂ concentrations. This example demonstrates that use of a combined PPI/B₁₂ product prevents the development of vitamin B₁₂ deficiency in patients who use PPI medications long-term by increasing serum vitamin B₁₂ concentrations.

A second subject, a 37 year old Caucasian male with no known history of gastrointestinal or pancreatic disease, vitamin deficiency or malabsorptive syndromes was also administered a composition comprising a PPI and vitamin B₁₂. He was not a vegetarian, and he took prescription simvastatin 20 mg daily. He had a baseline vitamin B₁₂ level of 303 pg/ml on Sep. 8, 2006 (laboratory reference range 200-1100 pg/ml). He began a vegetarian diet on September 8 and also began taking OTC omeprazole 20 mg daily on September 8. On September 9, he began taking 1000 mcg of oral vitamin B₁₂ within a capsule containing the vitamin B₁₂ and OTC omeprazole 20 mg daily. After 5 days of taking the combined B₁₂/OTC omeprazole, a repeat serum vitamin B₁₂ level was obtained. On Sep. 13. 2006 his serum vitamin B₁₂ level was 451 pg/ml (same lab as performed the first test, reference range 200-1100 pg/ml).

### Example 4

Patients and Setting

The study was conducted in a University-based, geriatric primary care setting, which serves a population largely over the age of 75 with multiple medical conditions and living in the community. Medical records were reviewed for patients 65 years of age or older. To be eligible, patients had to have a documented serum vitamin B₁₂ measurement between Jan. 1, 1990 and Dec. 31, 1997. Patients with a history of gastrectomy, partial gastrectomy, vagotomy, pancreatic insufficiency, pernicious anemia (positive Schilling test or anti-intrinsic factor antibodies), renal insufficiency (serum creatinine >2.0; possible accumulation of methylmalonic acid), or who were previously diagnosed with vitamin B₁₂ deficiency and were receiving B₁₂ therapy prior to Jan. 1, 1990, were excluded. These exclusions were made to avoid potentially confounding causes of vitamin B₁₂ deficiency. Also, patients with a low-normal serum vitamin B₁₂ (130-300 pg/mL, inclusive) and no assessments of methylmalonic acid (MMA) and homocysteine (HCYS) were excluded, because a portion of these patients would likely be found deficient (had MMA and HCYS been done) and misclassified as having normal B₁₂ status.

Data Collection

Standardized data collection forms were utilized to abstract information from medical records. Data collected included demographic information, serum vitamin B₁₂ studies, MMA and HCYS, serum folate, serum creatinine, mean corpuscular volume, history of gastrectomy or vagotomy, pancreatic insufficiency, documentation of pernicious anemia (abnormal Schillings test or presence of anti-intrinsic factor antibodies), documentation of H. pylori infection, multivitamin use, and vitamin B₁₂ therapy. Dates of use of prescription H2RA and/or PPI, including dose, regimen, and refill history, also were collected.

Vitamin B₁₂-deficient cases

Patients were considered to be vitamin B₁₂ deficient if their serum level was less than 130 pg/mL (laboratory reference range 130 to 770 pg/mL). Patients with low-normal serum vitamin B₁₂ concentrations (130 to 300 pg/mL, inclusive) were also considered deficient if MMA and HCYS were elevated (laboratory reference range 73-271 nmol/L for MMA and 5.1-13.9 pmol/L for HCYS). Fifty-three patients met the above criteria and constituted the vitamin B₁₂ deficient case group.

Vitamin B₁₂ Normal Controls

Patients with serum B₁₂ concentrations >300 pg/mL and patients with low-normal serum B₁₂ concentrations (130 to 300 pg/mL, inclusive) plus normal MMA/HCYS concentrations were considered to have normal B₁₂ status. Patients from the study were excluded if vitamin B₁₂ levels were in the low-normal range and no MMA/HCYS analysis had been done to ensure proper group assignment. From among the total pool of eligible control patients, a set of controls was frequency matched by age (within 1 year) and sex at a ratio of 4:1 to case patients using a computer-generated random selection algorithm. A total of 212 patients with normal vitamin B₁₂ status constituted the control group. H2RA, PPI, and MVI use

Information regarding the use of prescription H2RA and PPI was collected from medication flow sheets, progress notes, and by review of medication refill authorizations documented in a dated running log in the medical record. For validation of medical record data, computerized pharmacy records were obtained for a sample of patients who purchased their prescription medications where possible. Patients were categorized into one of four groups, a priori: no H2RA/PPI use, past use, short-term current use, or chronic current use. Patients who had a history of H2RA/PPI use but were not prescribed these medications at the time (or within 30 days) of vitamin B₁₂ studies being performed were classified as past H2RA/PPI users. Patients who were current H2RA/PPI users for <12 months at the time B₁₂ studies were performed were classified as short-term current H2RA/PPI users. Patients who had been taking H2RA or PPI for >12 months at the time B₁₂ studies were performed were considered chronic current H2RA/PPI users for the analysis. Information concerning over-the-counter use of H2RA was collected when available, but was not used for the analysis due to inconsistent documentation. Additionally, these products had been marketed for OTC use for only a short period of time when the study was initiated and the majority of serum vitamin B₁₂ studies had been obtained prior to FDA approval for OTC use. Multivitamin use was collected from medication histories and medication reviews documented in the progress notes.

Statistical Analysis

Descriptive statistics were used to characterize patient demographics. Student's t-test was used to compare means for continuous variables between patients with chronic use and those without (i.e., short-term use, past use, or no use). Pearson's chi-square test for independence was used to evaluate the bivariate association between categorical variables. Multivariable logistic regression analysis was used to calculate the odds ratio (with 95% confidence interval) of chronic acid suppressant use for B₁₂-deficient cases (vs. controls), both in unadjusted form and adjusting for age, gender, multivitamin use, and H. pylori infection. Comparisons with P-values of less than 0.05 (two-sided) were considered statistically significant. All statistical analyses were performed using SPSS version 11.0.

Results

A total of 1,305 patient records were reviewed for the study. Eight hundred thirty-eight (64.2%) had at least one documented serum vitamin B₁₂ concentration between Jan. 1, 1990 and Dec. 31, 1997. Sixteen patients with a history of gastrectomy or vagotomy, seven patients with documented pernicious anemia, and two patients with pancreatic insufficiency were excluded from the analysis. Fifteen of these 25 patients were vitamin B₁₂ deficient. Ninety-six patients were excluded from the study because of low-normal serum vitamin B₁₂ studies without assessments of MMA and HCYS. Fifty-three patients (7.4% of eligible subjects) were B₁₂ deficient as defined above (28 patients with serum B₁₂<130 pg/mL; 25 patients with elevated metabolites). Of the 28 patient with serum vitamin B₁₂ concentrations <130 pg/mL, 12 patients were <75 pg/mL, seven patients were between 76 and 100 pg/mL, and nine were between 101 and 129 pg/mL. Three of these 28 patients also had serum testing for MMA, and all had elevated concentrations of MMA, confirming deficiency. Control patients (n=212) were frequency matched by age and sex from the remaining 664 patients at a ratio of 4:1 to case patients (Table 1).

Computerized pharmacy records were obtained for a sample of 38 patients to validate medical record H2RA/PPI exposure data. Complete computerized pharmacy records for 36 of these patients were obtained. There was strong concordance between medical record data and computerized data. Eight of the 36 patients had been prescribed either an H2RA or a PPI. Using the number of prescriptions filled (standardized to reflect 1 month of therapy) and dividing by the number of months of therapy obtained from medical record review gave an mean value of 0.89 (range 0.81-1.02). Three of the eight patients had been classified as past users, and none of the three had filled prescriptions for either an H2RA or PPI within the month prior to vitamin B₁₂ testing. Similarly, all five of the current users had filled prescriptions for an H2RA or PPI within the month prior to vitamin B₁₂ testing.

TABLE 1
Characteristics of case and control subjects

| | Cases | Controls |
|---|---|---|
| Characteristic | (n = 53) | (n = 212) |
| Age, years | | |
| Mean (SD) | 82.19 (6.82) | 82.14 (6.62) |
| Age Category (n, %) | | |
| 65-74 | 5 (9.4) | 20 (9.4) |
| 75-84 | 31 (58.5) | 132 (62.3) |
| ≧85 | 17 (32.1) | 60 (28.3) |
| Gender (n, %) | | |
| Female | 34 (64.2) | 136 (64.2) |
| Male | 19 (35.8) | 76 (35.8) |
| Serum Creatinine, mg/dL | | |
| Mean (SD) | 1.09 (0.42) | 1.06 (0.34) |
| MVI Use (n, %) | | |
| Yes | 20 (37.7) | 102 (48.1) |
| No | 33 (62.3) | 110 (51.9) |
| Thyroid diagnosis (n, %) | | |
| Yes | 21 (39.6) | 41 (19.3) |
| No | 32 (60.4) | 171 (80.7) |
| H. pylori diagnosis (n, %) | | |
| Yes | 0 (0.0) | 4 (1.9) |
| No | 53 (100.0) | 208 (98.1) |
| B-12 Treatment (n, %) | | |
| Yes | 52 (98.1) | 7 (3.3) |
| No | 1 (1.9) | 205 (96.7) |
| Past AST Use (n, %) | | |
| Yes | 10 (18.9) | 22 (10.4) |
| No | 43 (81.1) | 190 (89.6) |
| If Yes, class of AST drug | | |
| H2RA | 7 (70.0) | 17 (87.3) |
| PPI | 3 (30.0) | 5 (22.7) |
| Current AST Use (n, %) | | |
| Yes | 16 (30.2) | 42 (19.8) |
| No | 37 (69.8) | 170 (80.2) |
| If Yes, class of AST drug | | |
| H2RA | 12 (75.0) | 34 (81.0) |
| PPI | 4 (25.0) | 8 (19.0) |
| Short-term current AST use (n, %) | | |
| Yes | 9 (17.0) | 35 (16.5) |
| No | 44 (83.0) | 177 (83.5) |
| If Yes, class of AST drug | | |
| H2RA | 6 (66.7) | 28 (80.0) |

Twenty-six case patients had either used H2RA/PPI in the past (n=10) or were current H2RA/PPI users (n=16). Of the current users, seven had been prescribed these medications for ≧12 months (mean duration 49±23 months). In the control group, 64 patients had either used H2RA/PPI in the past (n=22) or were current H2RA/PPI users (n=42). Of the current users in the control group, seven had been prescribed one of these medications for >12 months (mean duration 30±15 months). In the unadjusted analysis, vitamin B₁₂ deficiency was not associated with past use of H2RA/PPI (OR 2.01; 95% CI 0.89-4.35) or short term current use of H2RA/PPI (OR 1.03; 95% CI 0.46-2.31). Vitamin B₁₂ deficiency was significantly associated with chronic current H2RA/PPI use (OR 4.46; 95% CI 1.49-13.33) (Table 2 and the FIGURE). Adjustment for age, gender, multivitamin use, and H. pylori infection did not significantly change this finding (OR 4.45; 95% CI 1.47-13.34).

TABLE 2
Crude and adjusted odds ratios of B-12 deficiency^{a}

| | Crude odds ratio | Adjusted odds ratio |
|---|---|---|
| Characteristic | (95% CI) | (95% CI) |
| Past AST use | 2.01 (0.89-4.35) | 2.00 (0.87-4.37) |
| Current AST use | 1.75 (0.89-3.44) | 1.75 (0.88-3.43) |
| Short-term current AST use | 1.03 (0.46-2.31) | 1.01 (0.47-2.28) |
| Chronic current AST use | 4.46 (1.49-13.33) | 4.45 (1.47-13.34) |
| MVI use | 0.65 (0.35-1.21) | 0.78 (0.44-1.41) |
| H. pylori diagnosis | 0.43 (0.02-8.17) | 0.54 (0.06-8.26) |
| Thyroid diagnosis | 2.74 (1.43-5.23) | 2.62 (1.35-5.21) |
| ^{a}CI indicates confidence interval. Adjusted odds ratios and 95% CIs are based on multivariable logistic regression models including all the factors listed plus age and gender. The past, current, short-term current, and chronic current definitions of AST use were calculated in separate models. Values for the other variables listed came from the model containing chronic current AST use. Referent categories are non use of AST for indicated | | |
| # time periods, no use of MVI, no diagnosis of thyroid disorder, and no diagnosis of H. pylori. | | |

The foregoing discussion of the invention has been presented for purposes of illustration and descriptions. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references disclosed herein are incorporated by reference in their entirety.

What is claimed:

1. A composition comprising a mixture of vitamin B₁₂ and a gastrointestinal acid reducing agent.

2. The composition of claim 1, wherein vitamin B₁₂ is not bound to protein.

3. The composition of claim 1, wherein the gastrointestinal acid reducing agent comprises a gastrointestinal acid secretion reducing agent.

4. The composition of claim 3, wherein the gastrointestinal acid secretion reducing agent is selected from the group consisting of a histamine2-receptor antagonist (H2RA), a proton-pump inhibitor (PPI), and a combination thereof.

5. The composition of claim 4, wherein the PPI comprises omeprazole, lansoprasole, pantoprazole, rabeprazole, or a combination thereof.

6. The composition of claim 4, wherein the H2RA comprises cimetidine, ranitidine, famotidine, nizatidine, or a combination thereof.

7. The composition of claim 1, wherein the gastrointestinal acid reducing agent comprises a gastrointestinal acid neutralizing agent.

8. The composition of claim 7, wherein the gastrointestinal acid neutralizing agent comprises a bicarbonate.

9. A method of reducing the risk of a vitamin B₁₂ deficiency in a subject having or in need of a gastrointestinal acid reduction treatment comprising administering to a subject in need of such treatment, a therapeutically effective amount of a composition comprising a mixture of vitamin B₁₂ and a gastrointestinal acid reducing agent.

10. The method of claim 9, wherein vitamin B₁₂ is not bound to protein.

11. The method of claim 9, wherein the subject having or in need of a gastrointestinal acid reduction treatment has a condition selected from the group consisting of gastrointestinal acid reflux disease, gastroesophageal reflux disease, peptic ulcer disease, duodenal ulcer disease, Zollinger-Ellison disease, Hiatal hernia, esophageal erosions, esophagitis, Barrett's esophagus and esophageal strictures.

12. The method of claim 9, wherein the gastrointestinal acid reducing agent comprises a gastrointestinal acid secretion reducing agent.

13. The method of claim 12, wherein the gastrointestinal acid secretion reducing agent comprises a histamine2-receptor antagonist (H2RA), a proton-pump inhibitor (PPI), or a combination thereof.

14. The method of claim 9, wherein the gastrointestinal acid reducing agent comprises a gastrointestinal acid neutralizing agent.

15. The method of claim 14, wherein the gastrointestinal acid neutralizing agent comprises a bicarbonate.

16. The method of claim 9, wherein the subject is a human.
